(19) United States
(12) Patent Application Publication
(10) Pub. No.: US 2010/0094328 A1
(43) Pub. Date: Apr. 15, 2010

(54) SYSTEM, DEVICE AND A METHOD FOR DILATING A STRICTURE IN A LUMEN AND FOR DETERMINING THE TRANSVERSE CROSS-SECTIONAL AREA OF A LUMEN OR CAVITY

(75) Inventors: John O'dea, County Galway (IE); Adrian Mchugh, County Galway (IE); Patrick Griffin, County Galway (IE)

Correspondence Address: SUGHRUE MION, PLLC, 2100 PENNSYLVANIA AVENUE, N.W., SUITE 800, WASHINGTON, DC, 20037, US

(73) Assignee: FLIP TECHNOLOGIES LIMITED, Dangan, Galway (IE)

(21) Appl. No.: 12/444,985

(22) PCT Filed: Jun. 27, 2008
(86) PCT No.: PCT/IE2008/000070

§ 371 Date: Jul. 30, 2009

(30) Foreign Application Priority Data
Jun. 27, 2007 (IE) S2007/0461
Nov. 19, 2007 (IE) S2007/0842

(51) Int. Cl. A61M 29/02

(52) U.S. Cl. 606/192

(57) ABSTRACT

A system (1) for dilating an occlusion (3) in an oesophagus (4) comprises a device (5) having a catheter (8) extending from a proximal end (9) to a distal end (10). An inflatable balloon (12) defining a hollow interior region (14) is located on the catheter (8) adjacent the distal end (10) thereof for dilating the occlusion (3). The balloon (12) is inflated with a saline solution by a pump (34) through an axial communicating bore (20) and radial communicating bores (21) in the catheter (8). A pair of stimulating electrodes (25) on the catheter (8) within the balloon (12) adjacent axially opposite ends (18,19) thereof receive stimulating voltage signals from a signal generator (43). Receiving electrodes (28) on the catheter (8) between the stimulating electrodes (25) produce resulting voltage signals in response to the stimulating current signal on the stimulating electrodes (25) which are indicative of the transverse cross-sectional area of the balloon (12) adjacent the receiving electrodes (28) when the balloon (12) is inflated with the saline solution. A microprocessor (35) determines the diameter of the balloon (12) at the receiving electrodes (28) from the resulting signals thereon, and displays a three-dimensional image (46) of the balloon (12) on a visual display screen (47) as well as the corresponding diameter values in windows (48) on the visual display screen (47). A surgeon observes an image of the balloon (12) which corresponds to an image of the occlusion (3) and adjacent portion of the oesophagus 4) during dilating of the occlusion (3).

The present invention relates to a system and a method for dilating a stricture in a lumen, and in particular, though not limited to a system and a method for dilating a stricture in a hollow biological organ, such as a hollow biological organ of a human subject, for example, a stricture or an occlusion in an oesophagus. The invention also relates to a device for dilating a stricture in a lumen, and in particular, though not limited to a device for dilating a stricture in a hollow biological organ, such as a hollow biological organ of a human subject, for example, a stricture or an occlusion in an oesophagus. The invention also relates to a system, a device and a method for determining one of the transverse cross-sectional area and the diameter of one of a lumen and a cavity, for example, though not limited to a lumen or cavity in the human or animal body.

In the treatment of a stricture or occlusion in a lumen, such as a hollow biological organ, for example, the oesophagus resulting from, for example, a cancerous growth, it is common to use a dilation balloon to expand the oesophagus. An example of such a dilation balloon is a dilation balloon sold by Boston Scientific under the trade mark CRE™ Fixed Wire Balloon Dilator. Such dilation balloons are inflated with a liquid, such as water, which causes the balloon to expand. Through use of an X-ray or other suitable imaging means, the diameter of the oesophagus is assessed. A balloon size is chosen, typically three inflation diameters per balloon are achievable and the chosen balloon is inserted into the oesophagus, and located in the occlusion. By inflating the balloon in the occlusion the occlusion is dilated. There are, however, problems associated with this technique. A recent study has shown that it is difficult to consistently determine the diameter to which the balloon expands for a given amount of inflating liquid introduced into the balloon. Not only does this result in the possibility of the balloon being under-inflated with the consequence of the occlusion being insufficiently dilated, but the opposite may also occur where the balloon is over-inflated, resulting in perforation of the oesophagus. This problem is not restricted to the use of such dilation balloons in the treatment of strictures and occlusions in the oesophagus but is a problem associated with many procedures in which a dilation balloon is used to dilate a stricture or occlusion in any lumen or hollow organ, for example, the intestine, the colon, an artery, a vein or the like.

There is therefore a need for a system and a method for addressing at least some of the problems of known systems and methods for dilating an occlusion, a stricture or the like in the oesophagus or other hollow organ of a subject, or a lumen, whether biological or otherwise. There is also a need for a device for dilating an occlusion, a stricture or the like in the oesophagus or other hollow organ of a subject, or a lumen, whether biological or otherwise.

There are also occasions when it is desirable to measure the transverse cross-sectional area or the diameter of a lumen or cavity for other procedures or purposes. For example, it may be desirable to measure the transverse cross-sectional area or the diameter of an oesophagus, a colon, a urethra, an artery, a vein or the like in a human or animal subject, or a cavity, for example, a cavity in the heart, the stomach and the like in a human or animal subject.

The present invention is directed towards providing a system and a method for dilating a stricture in a lumen, and the invention in particular is directed towards, although is not limited to a system and a method for dilating an occlusion, a stricture or the like in the oesophagus of a human or animal subject, or in any other lumen or hollow organ in a human or animal subject, as well as in any other lumen or hollow organ, biological or otherwise. The invention is also directed towards a device for dilating an occlusion, stricture or the like in the oesophagus of a human or animal subject, or in any other lumen or hollow organ in a human or animal subject, as well as in any other lumen or hollow organ, biological or otherwise. The invention is further directed towards providing a system, a device and a method for determining one of the transverse cross-sectional area and the diameter of one of a lumen or cavity.

According to the invention there is provided a device for dilating a stricture in a lumen, the device comprising a catheter extending between a proximal end and a distal end, an inflatable balloon defining a hollow interior region located on the catheter towards the distal end thereof with the catheter extending through the hollow interior region of the balloon, the balloon being adapted for locating in the lumen adjacent the stricture to dilate the stricture, a communicating means communicating the hollow interior region of the balloon with the exterior thereof for accommodating an inflating medium to and from the hollow interior region of the balloon, at least one stimulating electrode located in the hollow interior region of the balloon on one of an inner surface of the balloon and an outer surface of the catheter, and at least one receiving electrode located in the hollow interior region of the balloon on one of the inner surface of the balloon and the outer surface of the catheter axially spaced apart from the stimulating electrode, an electrical conducting means electrically coupled to the stimulating and receiving electrodes, and electrically accessible exteriorly of the hollow interior region of the balloon, for facilitating applying one of a stimulating voltage signal and a stimulating current signal to the at least one stimulating electrode via the electrical conducting means, and for facilitating reading one of a resulting voltage signal and a resulting current signal on the at least one receiving electrode via the electrical conducting means indicative of the transverse cross-sectional area of the balloon, so that the transverse cross-sectional area of the balloon can be monitored during inflating thereof with an electrical conducting medium for determining the transverse cross-sectional area of the stricture.

Preferably, a pair of axially spaced apart stimulating electrodes are provided, and each receiving electrode is located between the stimulating electrodes. Advantageously, a plurality of axially spaced apart receiving electrodes are provided for producing resulting signals indicative of the transverse cross-sectional area of the balloon at corresponding axially spaced apart locations. Advantageously, the stimulating and receiving electrodes are located on the catheter.

In one embodiment of the invention the communicating means extends through the catheter. Preferably, the communicating means comprises an axial communicating bore extending through the catheter from the proximal end thereof to the hollow interior region of the balloon. Advantageously, at least one radial communicating bore extends through the catheter from the axial communicating bore to the hollow interior region of the balloon for communicating the hollow interior region of the balloon with the axial communicating bore.

In another embodiment of the invention the electrical conducting means comprises a plurality of mutually insulated electrically conductive wires extending through the catheter coupled to respective ones of the stimulating and receiving electrodes. Preferably, the electrically conductive wires extend through an axial wire accommodating bore extending through the catheter from the proximal end thereof to the stimulating and receiving electrodes. Advantageously, at least one radial wire accommodating opening extends through the catheter to the axial wire accommodating bore for accommodating the wires from the axial wire accommodating bore to the respective stimulating and receiving electrodes. Ideally, a plurality of radial wire accommodating openings are provided for accommodating respective ones of the wires from the axial wire accommodating bore to the respective electrodes.

In one embodiment of the invention each electrode is provided by an electrically conductive band. Advantageously, the electrically conductive band of each electrode extends completely around the catheter.

In one embodiment of the invention the balloon is an elongated balloon. Preferably, the balloon when inflated is of circular transverse cross-section. Advantageously, the balloon when inflated is of cylindrical configuration. Ideally, the balloon defines a central balloon axis substantially coinciding with a central longitudinal axis of the catheter.

In another embodiment of the invention a plurality of inflatable balloons are located axially along the catheter towards the distal end thereof. Preferably, at least one stimulating electrode and at least one receiving electrode axially spaced apart from the at least one stimulating electrode are located in the hollow interior region of each balloon. Advantageously, the balloons are located adjacent each other. Ideally, the respective balloons are inflatable independently of each other.

In one embodiment of the invention at least one of the balloons is of axial length different to the axial length of another one of the balloons.

In another embodiment of the invention a central one of the balloons is of axial length longer than the other balloons. Preferably, the axial length of the balloons increases progressively from the respective outer ones of the balloons to the central one of the balloons.

In another embodiment of the invention a central one of the balloons is of axial length shorter than the axial length of the other balloons. Preferably, the axial length of the balloons progressively increases from the central one of the balloons to the respective outer ones thereof.

In another embodiment of the invention at least one of the balloons is of different transverse cross-sectional area when inflated to the transverse cross-sectional area of another one of the balloons when inflated.

In another embodiment of the invention a central one of the balloons is of transverse cross-sectional area when inflated which is greater than the transverse cross-sectional area of the other balloons when inflated. Preferably, the transverse cross-sectional area of the balloons when inflated increases progressively from the respective outer balloons to the central one of the balloons.

In another embodiment of the invention a central one of the balloons is of lesser transverse cross-sectional area when inflated than the transverse cross-sectional area of the other balloons when inflated. Preferably, the transverse cross-sectional area of the balloons when inflated increases from a central one of the balloons to the respective outer ones thereof.

In a further embodiment of the invention the transverse cross-sectional shape of at least one of the balloons when inflated is different to the transverse cross-sectional shape of another one of the balloons when inflated.

In one embodiment of the invention the device is adapted for dilating a stricture in a hollow organ.

In another embodiment of the invention the device is adapted for dilating a stricture in a hollow biological organ.

In a further embodiment of the invention the device is adapted for dilating an occlusion in a hollow biological organ.

In a still further embodiment of the invention the device is adapted for dilating an occlusion in an oesophagus.

The invention also provides a system for dilating a stricture in a lumen, the system comprising a catheter extending between a proximal end and a distal end, an inflatable balloon defining a hollow interior region located on the catheter towards the distal end thereof with the catheter extending through the hollow interior region of the balloon, the balloon being adapted for locating in the lumen adjacent the stricture, an inflating means for inflating the balloon with an inflating medium to dilate the stricture, a communicating means communicating the inflating means with the hollow interior region of the balloon for accommodating the inflating medium between the inflating means and the hollow interior region of the balloon, at least one stimulating electrode located in the hollow interior region of the balloon on one of an inner surface of the balloon and an outer surface of the catheter for receiving one of a stimulating voltage signal and a stimulating current signal, at least one receiving electrode located in the hollow interior region of the balloon on one of the inner surface of the balloon and the outer surface of the catheter axially spaced apart from the stimulating electrode for producing a resulting signal indicative of the transverse cross-sectional area of the balloon adjacent the at least one receiving electrode in response to the one of the stimulating voltage signal and stimulating current signal when the balloon is inflated with an electrically conductive medium, a signal generator for generating the one of the stimulating voltage signal and the stimulating current signal, a control means for operating the signal generator for producing the one of the stimulating voltage signal and the stimulating current signal and for reading the resulting signal on the at least one receiving electrode in response to the one of the stimulating voltage signal and the stimulating current signal being applied to the at least one stimulating electrode, an electrically conductive means electrically coupling the signal generator to the at least one stimulating electrode for applying the one of the stimulating voltage signal and the stimulating current signal thereto, and for electrically coupling the control means to the at least one receiving electrode for reading the resulting signal indicative of the transverse cross-sectional area of the balloon therefrom, and a display means for displaying an image representative of the balloon derived from the resulting signal read by the control means from the at least one receiving electrode.

In one embodiment of the invention the communicating means extends through the catheter.

In another embodiment of the invention the inflating means is coupled to the axial communicating bore adjacent the proximal end of the catheter by a conduit.

Preferably, the electrical conducting means comprises a plurality of mutually insulated electrically conductive wires extending through the catheter coupled to respective ones of the stimulating and receiving electrodes. Advantageously, the electrically conductive wires extend through an axial wire accommodating bore extending through the catheter from the proximal end thereof to the stimulating and receiving electrodes. Ideally, at least one radial wire accommodating opening extends through the catheter to the axial wire accommodating bore for accommodating the wires from the axial wire accommodating bore to the respective stimulating and receiving electrodes. Preferably, a plurality of radial wire accommodating bores are provided for accommodating respective ones of the wires from the axial wire accommodating bore to the respective electrodes.

In one embodiment of the invention the signal generator is coupled to the stimulating electrodes by respective corresponding ones of the wires.

In another embodiment of the invention the control means is coupled to the receiving electrodes by respective corresponding ones of the wires.

Preferably, a pressure monitoring means is provided for monitoring the pressure of the inflating medium in the balloon. Advantageously, the control means reads signals from the pressure monitoring means. Ideally, the control means is responsive to signals from the pressure monitoring means for controlling the operation of the inflating means.

In another embodiment of the invention the inflating means comprises a pump.

In another embodiment of the invention the control means is responsive to the resulting signals from the respective receiving electrodes for computing diameter values of the balloon adjacent the respective receiving electrode, and the respective diameters are displayed on the visual display means.

In one embodiment of the invention the system is adapted for dilating a stricture in a hollow organ.

In another embodiment of the invention the system is adapted for dilating a stricture in a hollow biological organ.

In a further embodiment of the invention the system is adapted for dilating an occlusion in a hollow biological organ.

In a still further embodiment of the invention the system is adapted for dilating an occlusion in an oesophagus.

The invention also provides a method for dilating a stricture in a lumen, the method comprising providing a catheter extending between a proximal end and a distal end, providing an inflatable balloon defining a hollow interior region located on the catheter towards the distal end thereof with the catheter extending through the hollow interior region of the balloon, providing a communicating means communicating the hollow interior region of the balloon with the exterior thereof for accommodating an inflating medium to and from the hollow interior region of the balloon, providing at least one stimulating electrode located in the hollow interior region of the balloon on one of an inner surface of the balloon and an outer surface of the catheter and at least one receiving electrode located in the hollow interior region of the balloon on one of the inner surface of the balloon and the outer surface of the catheter axially spaced apart from the stimulating electrode, providing an electrically conducting means electrically coupled to the stimulating and receiving electrodes, and electrically accessible exteriorly of the hollow interior region of the balloon, the method further comprising entering the distal end of the catheter with the balloon thereon into the lumen until the balloon is located in the lumen with the balloon substantially centrally axially located relative to the stricture, inflating the balloon with an electrically conductive inflating medium, applying one of a stimulating voltage signal and a stimulating current signal to each stimulating electrode and reading a resulting signal on the at least one of the receiving electrodes adjacent the stricture for determining the transverse cross-sectional area of the balloon during inflating thereof, and continuing inflating of the balloon until the transverse cross-sectional area of the balloon corresponds to a desired transverse cross-sectional area to which the stricture is to be dilated.

Preferably, the balloon is initially partially inflated on being inserted in the lumen, and is urged along the lumen in the partially inflated state for identifying the stricture from the resulting signal read from the at least one receiving electrode.

Alternatively, the balloon is urged along the lumen until the balloon is located in the general area of the stricture, and when the balloon is in the general area of the stricture, the balloon is incrementally urged along the lumen, and on each incremental movement of the balloon the balloon is inflated with the electrically conductive inflating medium and subsequently deflated, and while being inflated, the one of the stimulating voltage signal and the stimulating current signal is applied to the at least one stimulating electrode, and the resulting signal is read from the at least one receiving electrode for determining when the balloon is axially centrally located relative to the stricture.

In one embodiment of the invention the desired transverse cross-sectional area to which the stricture is to be dilated is determined as a function of the transverse cross-sectional area of the lumen adjacent the stricture. Preferably, the desired transverse cross-sectional area to which the stricture is to be dilated is determined as a percentage function of the transverse cross-sectional area of the lumen adjacent the structure.

In one embodiment of the invention a plurality of inflatable balloons are located on the catheter towards the distal end thereof, and the respective balloons are inflated with the electrically conductive medium, and the one of the stimulating voltage signal and the stimulating current signal are applied to the respective stimulating electrodes in the respective balloons and the resulting signals from the corresponding receiving electrodes are read for determining the diameter of the respective balloons during inflating thereof, and the one or the ones of the balloons adjacent the stricture is inflated until the transverse cross-sectional area thereof corresponds with the desired transverse cross-sectional area to which the stricture is to be dilated.

Advantageously, a central one of the balloons is axially centrally located relative to the stricture. Preferably, respective ones of the balloons located at axial opposite ends of the stricture are inflated for determining the transverse cross-sectional area of the lumen adjacent the respective axial opposite ends of the stricture, and the one or the ones of the balloons adjacent the stricture is inflated to a diameter which is a function of the diameter of the lumen adjacent the stricture for dilating the stricture to the desired diameter. Advantageously, the one or the ones of the balloons adjacent the stricture is inflated to a diameter which is a percentage function of the diameter of the lumen adjacent the stricture for dilating the stricture to the desired diameter.

In one embodiment of the invention the respective balloons are independently inflatable, and the balloons are independently inflated relative to each other.

Preferably, an image representative of each balloon is displayed on a visual display screen.

Advantageously, approximate values of the diameter of the respective balloons adjacent the corresponding receiving electrodes are determined and displayed on the visual display screen.

In one embodiment of the invention the method is adapted for dilating a stricture in a hollow organ.

In another embodiment of the invention the method is adapted for dilating a stricture in a hollow biological organ.

In a further embodiment of the invention the method is adapted for dilating a stricture in an oesophagus.

In a still further embodiment of the invention the method is adapted for dilating an occlusion in an oesophagus.

In a still further embodiment of the invention the method is adapted for dilating a stricture in a biological lumen.

The invention further provides a device for determining one of the transverse cross-sectional area and the diameter of one of a lumen and a cavity at a plurality of axially spaced apart locations, the device comprising a catheter extending between a proximal end and a distal end, a plurality of inflatable balloons defining respective hollow interior regions located on the catheter towards the distal end thereof with the catheter extending through the hollow interior regions of the respective balloons, at least one stimulating electrode located within the hollow interior region of each balloon on one of an outer surface of the catheter and an inner surface of the balloon for receiving one of a stimulating voltage signal and a stimulating current signal, at least one receiving electrode located within the hollow interior region of each balloon on one of the outer surface of the catheter and the inner surface of the balloon axially spaced apart from the corresponding at least one stimulating electrode for producing a resulting signal indicative of the one of the transverse cross-sectional area and the diameter of the balloon adjacent the at least one corresponding receiving electrode in response to the corresponding one of the stimulating voltage signal and the stimulating current signal when the corresponding balloon is inflated with an electrically conductive inflating medium.

In one embodiment of the invention respective electrical conducting means are electrically coupled to the stimulating and receiving electrodes of the respective balloons so that the corresponding stimulating and receiving electrodes are electrically addressable exteriorly of the corresponding balloons.

Further the invention provides a system for determining one of the transverse cross-sectional area and the diameter of one of a lumen and a cavity at a plurality of axially spaced apart locations, the system comprising the device according to the invention, and at least one inflating means for inflating the respective balloons with an inflating medium, a signal generator for applying one of a stimulating voltage signal and a stimulating current signal to the at least one stimulating electrode of the respective balloons, and a control means for reading the resulting signals on the receiving electrodes of the respective balloons in response to the respective one of the stimulating voltage signal and the stimulating current signal when the corresponding balloons are inflated with the electrically conductive inflating medium, and for determining the one of the transverse cross-sectional area and the diameter of the respective balloons adjacent the corresponding receiving electrodes.

Additionally the invention provides a system for determining one of the transverse cross-sectional area and the diameter of one of a lumen and a cavity at a plurality of axially spaced apart locations, the system comprising a catheter extending between a proximal end and a distal end, a plurality of inflatable balloons defining respective hollow interior regions located on the catheter towards the distal end thereof with the catheter extending through the hollow interior regions of the respective balloons, at least one stimulating electrode located within the hollow interior region of each balloon on one of an outer surface of the catheter and an inner surface of the balloon for receiving one of a stimulating voltage signal and a stimulating current signal, at least one receiving electrode located within the hollow interior region of each balloon on one of the outer surface of the catheter and the inner surface of the balloon axially spaced apart from the corresponding at least one stimulating electrode for producing a signal indicative of the one of the transverse cross-sectional area and the diameter of the balloon adjacent the at least one receiving electrode in response to the corresponding one of the stimulating voltage signal and the stimulating current signal when the balloon is inflated with an electrically conductive inflating medium, at least one inflating means for inflating the respective balloons with the electrically conductive medium, a signal generator for applying the one of the stimulating voltage signal and the stimulating current signal to the stimulating electrodes of the respective balloons, a control means for reading the resulting signals from the receiving electrodes of the respective balloons in response to the corresponding one of the stimulating voltage signal and the stimulating current signal when the corresponding balloon is inflated with electrically conductive medium, and for determining the one of the transverse cross-sectional area and the diameter of the respective balloons adjacent the respective receiving electrodes and for outputting respective signals indicative of the respective ones of the transverse cross-sectional area and the diameter of the respective balloons at the respective receiving electrodes.

In one embodiment of the invention a display means is provided for displaying an image representative of the respective balloons in response to the signals produced by the control means which are indicative of the one of the transverse cross-sectional area and the diameter of the respective balloons adjacent the respective receiving electrodes.

Preferably, the diameter of the balloons adjacent the respective receiving electrodes are displayed on the display means along with the image representative of the balloons.

In one embodiment of the invention a monitoring means is provided for monitoring the pressure to which the respective balloons are inflated, and the control means reads signals from the pressure monitoring means. Preferably, the control means is responsive to signals read from the pressure monitoring means for controlling the operation of inflating means for inflating the respective balloons. Advantageously, the control means is responsive to the pressures read from the pressure monitoring means for determining when the respective balloons have been inflated to a state abutting an inner wall defining the one of the lumen and the cavity.

The invention also provides a method for determining one of the transverse cross-sectional area and the diameter of one of a lumen and a cavity at a plurality of axially spaced apart locations, the method comprising providing a catheter extending between a proximal end and a distal end, providing a plurality of inflatable balloons defining respective hollow interior regions on the catheter towards the distal end thereof with the catheter extending through the hollow interior regions of the respective balloons, providing at least one stimulating electrode located within the hollow interior region of each balloon on one of an outer surface of the catheter and an inner surface of the balloon for receiving one of a stimulating voltage signal and a stimulating current signal, providing at least one receiving electrode located within the hollow interior region of each balloon on one of the inner surface of the catheter and the inner surface of the balloon axially spaced apart from the corresponding at least one stimulating electrode for producing a resulting signal indicative of the one of the transverse cross-sectional area and the diameter of the balloon adjacent the at least one receiving electrode in response to the corresponding one of the stimulating voltage signal and the stimulating current signal when the balloon is inflated with an electrically conductive inflating medium, the method further comprising inflating the respective balloons to fill the portion of the one of the lumen and the cavity adjacent where the one of the transverse cross-sectional area and the diameter thereof are to be determined so that the respective balloons abut a wall of the one of the lumen and the cavity, applying the one of the stimulating voltage signal and the stimulating current signal to the stimulating electrodes of the respective balloons, reading the resulting signals on the receiving electrodes, and determining the one of the transverse cross-sectional area and the diameter of the balloons adjacent the respective receiving electrodes from the resulting signals.

Preferably, an image representative of the inflated balloons is produced from the resulting signals read from the receiving electrodes of the respective balloons and displayed on a visual display screen.

Advantageously, the diameter values of the respective balloons at the locations adjacent the receiving electrodes are displayed on the visual display screen along with the image representative of the balloons.

The advantages of the invention are many. A particularly important advantage of the invention is that the balloon for dilating the stricture can be readily placed and axially centrally aligned with the stricture in the lumen without a requirement for any X-ray, ultrasonic, fluoroscopy or other forms of imaging which are required for known systems and devices. By virtue of the fact that the transverse cross-sectional area of the balloon is continuously determined as the balloon is being inflated, and furthermore, by virtue of the fact that the pressure in the balloon is monitored during inflating thereof, the diameter of the balloon when the balloon first comes into abutting engagement with the stricture and the lumen adjacent the stricture can be readily determined. Since the transverse cross-sectional area of the balloon is continuously monitored during inflating thereof, an image of the balloon on a visual display screen can be provided and be continuously updated. Additionally, the diameter of the balloon at respective axially spaced apart locations which correspond to the receiving electrodes can be displayed on the visual display screen, along with the image of the balloon. Thus, a surgeon, doctor or other paramedic using the device can firstly, determine the diameter of the lumen adjacent the stricture, can determine the diameter of the stricture, and both of these diameters can be determined without the need for any X-ray, ultrasonic, fluoroscopy or other such forms of imaging. Once the diameter to which the stricture is to be dilated has been determined, the stricture can then be readily dilated to the desired diameter without any requirement of X-ray, ultrasonic, fluoroscopy or other such forms of imaging, since the diameter of the stricture as it is being progressively dilated is displayed on the visual display screen to be observed by the doctor, surgeon or other paramedic. Additionally, by monitoring the pressure in the balloon, any danger of over-pressurising the balloon which could otherwise result in perforation of the balloon is avoided.

Accordingly, the system and the device according to the invention provides for both location and dilation of a stricture or an occlusion in a lumen, a hollow organ, vessel or the like without the need for X-ray, ultrasonic, fluoroscopy or other forms of imaging of the device to be made when it is being located in the lumen, organ or vessel adjacent the occlusion. Additionally, the use of the system, device and method according to the invention avoids the need for endoscopy.

The device, system and method according to the invention are particularly advantageous for determining the transverse cross-sectional area and diameter of a lumen or cavity, whether biological or otherwise. The device, system and method according to the invention provide a relatively accurate indication of the transverse cross-sectional area of a lumen or cavity, and provide a reasonably accurate indication of the diameter thereof.

The invention will be more clearly understood from the following description of some preferred embodiments thereof which are given by way of example only with reference to the accompanying drawings in which:

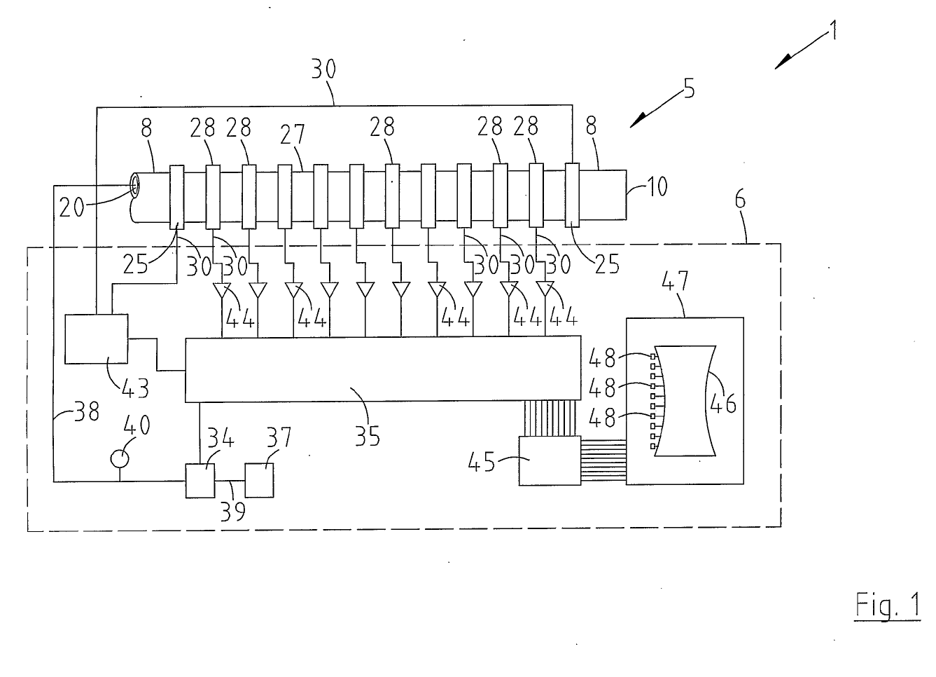

FIG. 1 is a block representation of a system according to the invention for dilating a stricture in a lumen, which comprises a device also according to the invention for dilating a stricture in a lumen,

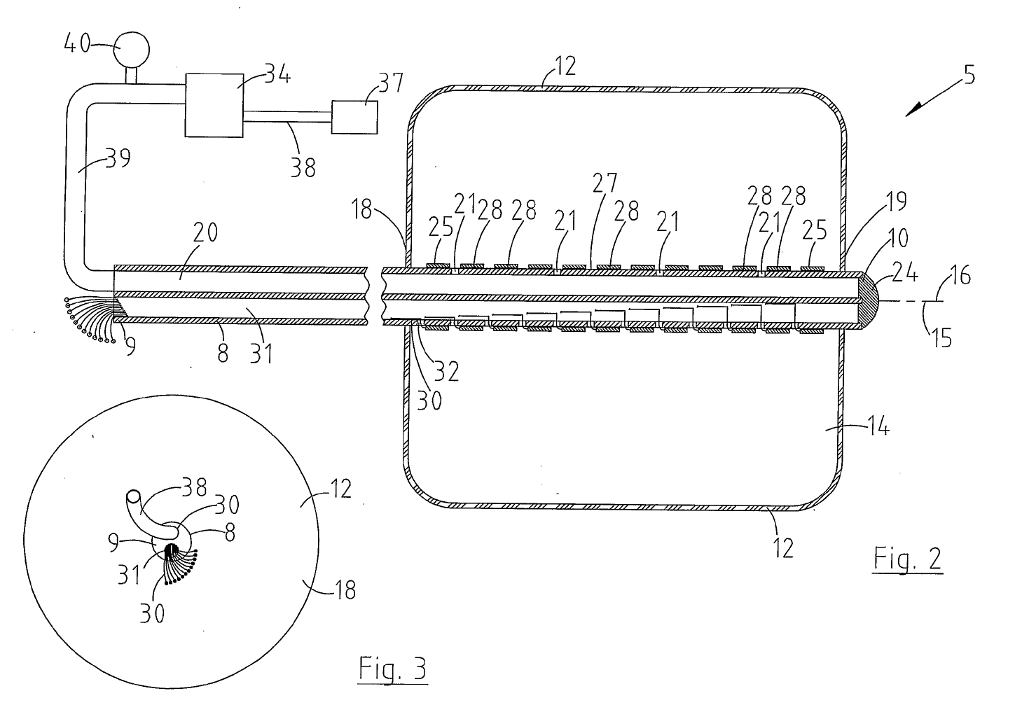

FIG. 2 is a transverse cross-sectional side elevational view of the device of FIG. 1 for dilating a stricture in a lumen,

FIG. 3 is an end elevational view of the device of FIG. 2,

FIG. 4 is a diagrammatic view of the device of FIG. 2 in use,

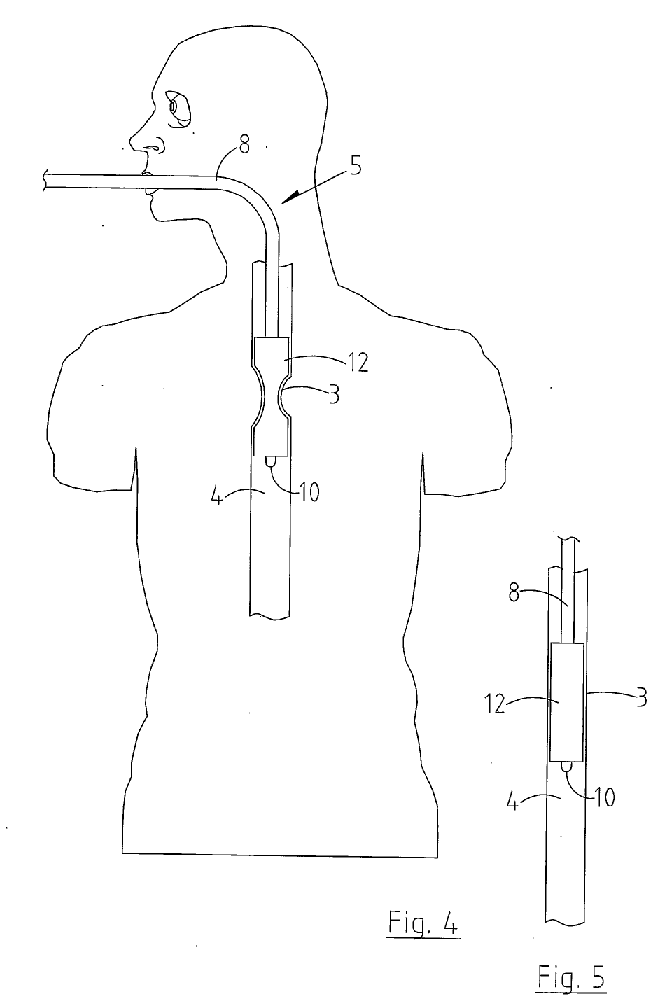

FIG. 5 is another diagrammatic view of the device of FIG. 2 in use,

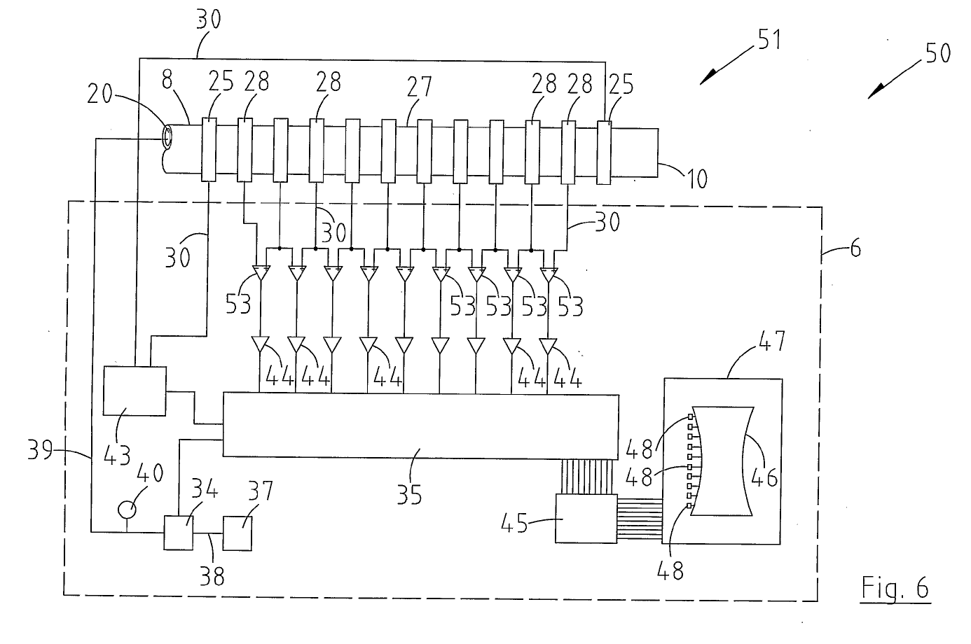

FIG. 6 is a block diagram of a system according to another embodiment of the invention for dilating a stricture in a lumen,

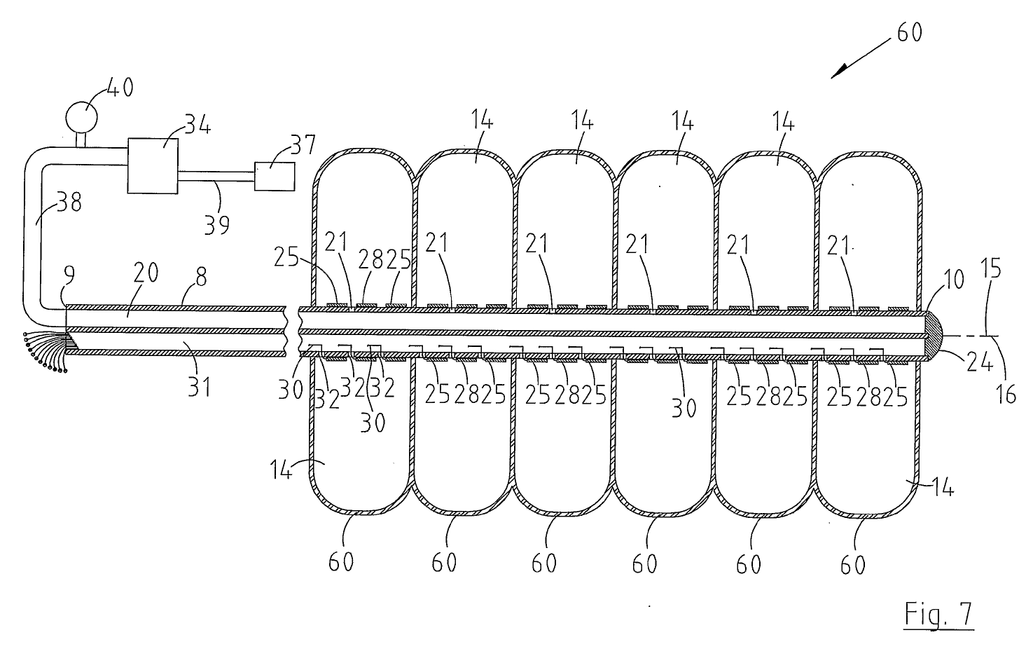

FIG. 7 is a transverse cross-sectional side elevational view of a device according to another embodiment of the invention for dilating a stricture in a lumen,

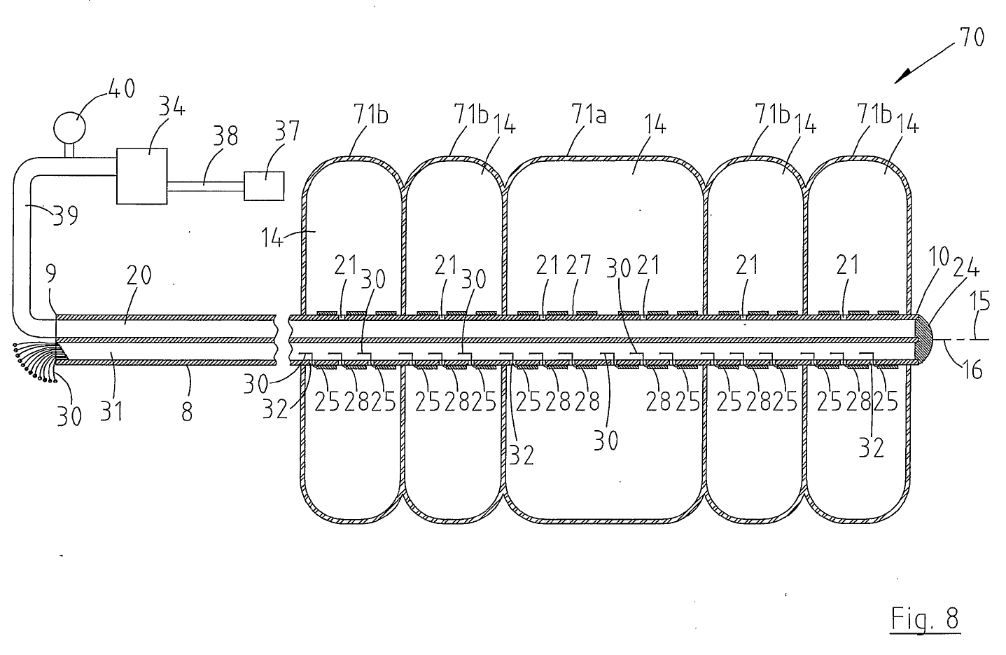

FIG. 8 is a transverse cross-sectional side elevational view of a device according to another embodiment of the invention for dilating a stricture in a lumen, and

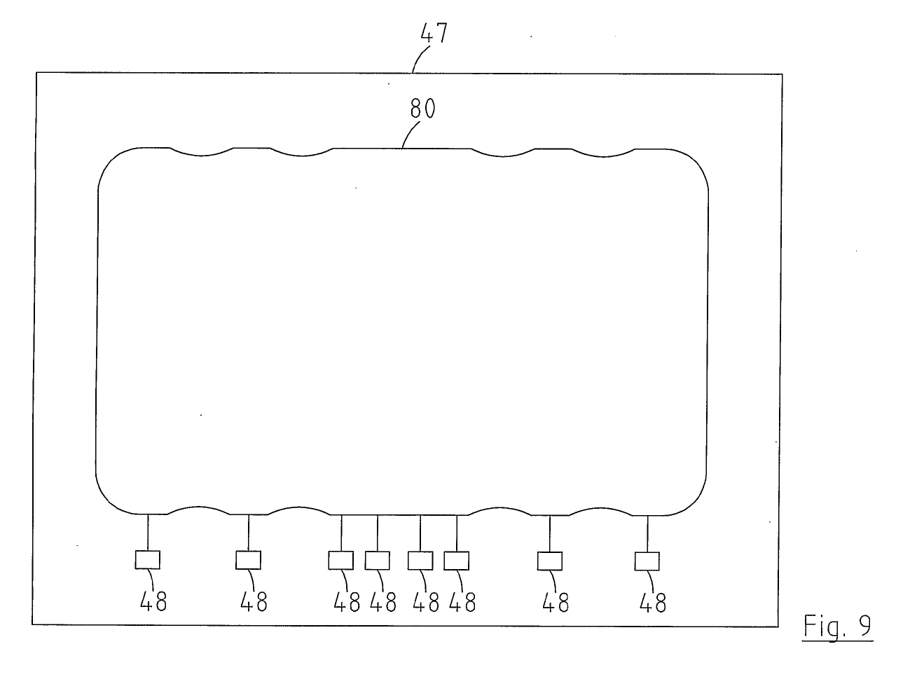

FIG. 9 is a view of an image representative of the balloons of the device of FIG. 8 inflated during use of the device in a method for determining the transverse cross-sectional area of a lumen or a cavity at a plurality of axially spaced apart locations.

Referring to the drawings and initially to FIGS. 1 to 5 thereof there is illustrated a system according to the invention indicated generally by the reference numeral 1 for dilating a stricture in a lumen, and in this embodiment of the invention the system 1 is particularly suitable for dilating an occlusion 3 in the oesophagus 4 of a human or animal subject, and in particular, a human subject. The system 1 comprises a device also according to the invention indicated generally by the reference numeral 5 for inserting into the oesophagus for dilating the occlusion 3. A control and analysing apparatus 6 controls operation of the system 1 and the device 5 as will be described below. Before describing the system 1 in further detail, the device 5 will first be described.

The device 5 comprises an elongated catheter 8 extending from a proximal end 9 to a distal end 10 for inserting into the oesophagus nasally or orally. An inflatable balloon 12 defining a hollow interior region 14 is located on the catheter 8 towards the distal end 10 thereof with the catheter 8 extending through the hollow interior region 14 thereof. In this embodiment of the invention the balloon 12 when inflated is of cylindrical configuration and defines a central longitudinally extending balloon axis 15 which coincides with a longitudinally extending central axis 16 of the catheter 8. The balloon 12 is sealably secured to the catheter 8 at its respective axially opposite ends 18 and 19, and is provided thereon for locating adjacent the occlusion 3 in the oesophagus 4 as will be described below.

A communicating means, in this embodiment of the invention an elongated axial communicating bore 20 extends longitudinally through the catheter 8 from the proximal end 9 to the distal end 10 thereof for accommodating an inflating medium, which in this case is an electrically conductive medium, preferably, a saline solution, from the control and analysing apparatus 6 for inflating the balloon 12, as will be described below. A plurality of radial communicating bores 21 extend radially through the catheter 8 within the hollow interior region 14 of the balloon 12 and communicate with the axial communicating bore 20 for accommodating the inflating medium between the axial communicating bore 20 and the hollow interior region 14 of the balloon 12 during inflating and deflating thereof. The catheter 8 terminates in a hemispherical plug 24 of epoxy resin which sealably closes the distal end of the axial communicating bore 20.

A pair of electrically conductive stimulating electrodes 25 for receiving one of a stimulating voltage signal and a stimulating current signal from the control and analysing apparatus 6 is located axially spaced apart on an outer surface 27 of the catheter 8 adjacent the respective axially opposite ends 18 and 19 of the balloon 12 and within the hollow interior region 14 thereof. In this embodiment of the invention the stimulating signal is a stimulating current signal of constant known current value. A plurality of electrically conductive receiving electrodes 28 in this case ten receiving electrodes 28 are located axially spaced apart on the outer surface 27 of the catheter 8 within the hollow interior region 14 of the balloon 12 and between and spaced apart from the stimulating electrodes 25. When the balloon 12 is inflated with the electrically conductive medium voltage signals appear on the receiving electrodes 28 in response to the stimulating current signal applied to the stimulating electrodes 25 which are indicative of the transverse cross-sectional area of the balloon 12 adjacent the respective receiving electrodes 28. The signals on the receiving electrodes 28 are read by the control and analysing apparatus 6 for determining the transverse cross-section area and the diameter of the balloon 12 at the locations corresponding to the receiving electrodes 28.

In this embodiment of the invention the stimulating electrodes 25 and the receiving electrodes 28 are provided by electrically conductive band electrodes which extend circumferentially around and are bonded to the catheter 8. The receiving electrodes 28 are located between the stimulating electrodes 25 and are equi-spaced apart from each other, and the spacing between the stimulating electrodes 25 and the adjacent receiving electrodes 28 in this embodiment of the invention is similar to the spacing between the receiving electrodes 28. However, the spacing between the receiving electrodes 28 may not be constant, and similarly, the spacing between the stimulating electrodes 25 and the adjacent receiving electrodes 28 may be the same or different to the spacing between the receiving electrodes 28.

An electrically conductive means through which the stimulating current signal is applied to the stimulating electrodes 25 from the control and analysing apparatus 6 and through which the resulting signals from the receiving electrodes 28 are delivered to the control and analysing apparatus 6 comprises a plurality of mutually insulated electrically conductive wires 30 which extend through a longitudinally extending axial wire accommodating bore 31 which extends through the catheter 8 from the proximal end 9 to the stimulating and receiving electrodes 25 and 28. A plurality of radial wire accommodating openings 32 extend radially through the catheter 8 and communicate with the axial wire accommodating bore 31 for accommodating the respective wires 30 from the axial wire accommodating bore 31 to the corresponding ones of the stimulating and receiving electrodes 25 and 28. A separate wire 30 is provided to each stimulating electrode 25 and each receiving electrode 28. The wires 30 extend from the axial wire accommodating bore 31 at the proximal end 9 thereof for coupling to the electronic control and analysing apparatus 6 as will be described below.

An inflating means comprising a pump 34 in the control and analysing apparatus 6 is operable under the control of a control means, namely, a microprocessor 35 for inflating and deflating the balloon 12 with the electrically conductive medium from a reservoir 32. In this case the reservoir 32 contains the saline solution. A conduit 38 couples the reservoir 37 to the pump 34, and a conduit 39 couples the pump 34 to the axially communicating bore 20 at the proximal end 9 of the catheter 8. The pump 34 is operable under the control of the microprocessor 35 for pumping the inflating medium from the reservoir 37 to the balloon 12, and for exhausting the inflating medium from the balloon 12 to the reservoir 37. A pressure monitoring means comprising a pressure sensor and a pressure gauge 40 monitors the pressure of the inflating medium in the conduit 39 for determining the pressure to which the balloon 12 is inflated. The microprocessor 35 reads signals from the pressure sensor and pressure gauge 40 for determining the pressure to which the balloon 12 is inflated, and also controls operation of the pump 34 in response to the pressure.

A constant current signal generator 43 in the control and analysing apparatus 6 is operable under the control of the microprocessor 35 for producing the constant current stimulating current signal. The stimulating current signal is applied to the stimulating electrodes 25 through a corresponding pair of the wires 30. The resulting voltage signals which appear on the receiving electrodes 28 are applied to respective corresponding analogue-to-digital converters 44 via the corresponding wires 30 from the receiving electrodes 28. Digital values of the respective resulting voltage signals are read by the microprocessor 35 from the respective analogue-to-digital converters 44. As discussed above, the resulting voltage signals appearing on the receiving electrodes 28 are indicative of the transverse cross-sectional area of the balloon 12 at axially spaced apart locations corresponding to the respective receiving electrodes 28. The microprocessor 35 is programmed for computing the transverse cross-sectional area of the balloon 12 at the respective axially spaced apart locations corresponding to the receiving electrodes 28 and approximate values of the corresponding diameters of the balloon 12.

The computed values of the diameter of the balloon 12 at the axially spaced apart locations corresponding to the receiving electrodes 28 are applied by the microprocessor 35 to a graphics processor 45 which develops a three dimensional image 46 which is representative of the inflated or partially inflated balloon 12, as the case may be, which is displayed on a visual display screen 47. The computed diameter values of the balloon 14 are displayed on the visual display screen 47 adjacent the image 46 in windows 48 along with the image 46 corresponding to the respective axially spaced apart locations. Thus, a doctor, surgeon or a paramedic operating the system 1 and the device 5 can readily identify from the image 46 on the visual display screen 47 when the balloon 12 is axially centrally located relative to the occlusion 3 in the oesophagus 4, and can also read the diameter of the occlusion 3 as well as the diameter of the oesophagus 4 at locations at respective axially opposite ends of the occlusion 3 from the corresponding windows 48 on the visual display screen 37.

The microprocessor 35 is programmed to compute the approximate diameter values of the balloon 12 at the axially spaced apart locations corresponding to the receiving electrodes 28 by determining the drop in voltage between the respective stimulating electrodes 25 and the adjacent receiving electrodes 28, as well as the voltage drop between adjacent ones of the respective receiving electrodes 28 in response to the stimulating current signal applied to the stimulating electrodes 25 when the balloon is inflated with the electrically conductive inflating medium, namely, the saline solution. The voltage drop between the stimulating electrodes 25 and the adjacent receiving electrodes, and the voltage drop between adjacent ones of the receiving electrodes 28 is a function of the electrical impedance of the saline solution between the respective stimulating electrodes 25 and the adjacent receiving electrodes 28 and the adjacent ones of the receiving electrodes 28, which in turn is a function of the volume of saline solution between the respective electrodes 25 and 28. Accordingly, both the transverse cross-sectional area and the diameter of the balloon 12 at the axially spaced apart locations adjacent the respective receiving electrodes 28 is a function of the respective voltage drops between adjacent ones of the receiving electrodes 28 and the stimulating and adjacent receiving electrodes 25 and 28.

In use, the distal end 10 of the catheter 8 with the balloon 12 deflated is entered into the oesophagus 4 either nasally or orally and is manoeuvred until the balloon 12 is in the general area of the occlusion 3. The balloon 12 is then inflated with the saline solution, and simultaneously with inflating the balloon 12 the stimulating current signal is applied to and maintained across the stimulating electrodes 25. The resulting voltages on the receiving electrodes 28 are read by the microprocessor 35 from the corresponding analogue-to-digital converters 44. The microprocessor 35 continuously computes the diameters of the balloon 12 at the axially spaced apart locations adjacent the receiving electrodes 28, and continuously updates the graphics processor 45 with the computed diameters. The graphics processor 45 in turn continuously updates the image 46 of the balloon 12 on the visual display screen 47 and also updates the diameter values of the balloon 12 displayed in the windows 48 on the visual display screen 47. This, thus, gives the surgeon, doctor or paramedic an indication of the location of the balloon 12 relative to the occlusion 3.

With the balloon 12 partially inflated, the balloon 12 is moved slowly along the oesophagus 4 adjacent the occlusion 3 while watching the image 46 on the visual display screen 47 in order to identify when the balloon 12 is axially centrally located relative to the occlusion 3. When the balloon 12 has been axially centrally located relative to the occlusion 3, the balloon 12 is further inflated with the saline solution for determining the diameter values of the oesophagus 4 at the respective axially opposite ends of the occlusion 3, which are read from the corresponding windows 48 on the visual display screen 47. The surgeon, doctor or paramedic then determines the desired diameter to which the occlusion 3 is to be dilated from the diameter values of the oesophagus 4 at the respective axially opposite ends of the occlusion 3.

During inflating of the balloon 12, the pressure of the saline solution in the balloon 12 is monitored by the pressure sensor and pressure gauge 40 for determining when the balloon 12 is in abutting engagement with the oesophagus 4 and the occlusion 3. During initial inflating of the balloon 12, the pressure of the saline solution in the balloon 12 remains substantially constant, or increases at a substantially constant rate. However, on the balloon 12 coming into tight abutment with the occlusion 3 and the oesophagus 4, the pressure within the balloon 12, if it had been substantially constant, commences to increase, or on the other hand, if the pressure in the balloon had been increasing at a substantially constant rate, the rate of increase in pressure of the saline solution in the balloon commences to increase more rapidly. This, thus, gives the surgeon an immediate indication as to when the balloon 12 is tightly abutting the occlusion 3 and the oesophagus 4.

It is when the balloon 12 is in abutting engagement with both the occlusion 3 and the oesophagus 4 that the diameter values of the oesophagus 4 adjacent the respective axially opposite ends of the occlusion 3 are read for determining the diameter to which the occlusion 3 is to be dilated. In general, it would be desirable to dilate the occlusion 3 to be of substantially similar diameter to the diameter of the oesophagus adjacent the respective axially opposite ends of the occlusion 3. However, in certain cases, it may be decided to dilate the occlusion 3 to a diameter less than the diameter of the oesophagus 4 on the respective axially opposite ends of the occlusion 3, and which would be a percentage of the diameter of the oesophagus at the respective axially opposite ends of the occlusion.

Once a determination of the desired diameter to which the occlusion 3 is to be dilated has been made, the balloon 12 is further inflated with the saline solution, and the stimulating current signal is maintained across the stimulating electrodes 25.

The microprocessor 35 continues to read the resulting voltage signals from the receiving electrodes 28 via the corresponding analogue-to-digital converters 44, and in turn continuously updates the diameter values of the balloon 12 at the respective axially spaced apart locations corresponding to the receiving electrodes 28, which are provided to the graphics processor 45. The graphics processor 45 in turn continuously updates the image 46 of the balloon 12 on the visual display screen 43, as well as the diameter values of the respective axially spaced apart locations in the windows 48. Inflating of the balloon 12 is continued until the occlusion 3 has been dilated to the desired diameter. However, during inflating of the balloon 12 to dilate the occlusion 3, the pressure of the saline solution in the balloon 12 is continuously monitored on the pressure sensor and pressure gauge 40 to avoid over-pressurising the balloon 12 which could otherwise result in perforation of the oesophagus 4.

Once the occlusion 3 has been dilated to the desired diameter, the pump 34 is activated for pumping the saline solution from the balloon 12 to the reservoir 47 for deflating the balloon 12. The catheter 8 with the balloon 12 deflated is then removed from the oesophagus 4.

An alternative method for axially centrally locating the balloon 12 relative to the occlusion 3 is to consecutively and alternatively inflate and deflate the balloon 12 with the saline solution as the balloon 12 is being incrementally urged along the oesophagus 3 until the balloon 12 is eventually located centrally axially relative to the occlusion 3. During the consecutive inflatings and deflatings of the balloon 12, the stimulating current signal is maintained on the stimulating electrodes 25, and the microprocessor 35 continuously monitors the voltage signals on the receiving electrodes 28, and the graphics processor 45 continuously updates the image 46 of the balloon 12 on the visual display screen 47 along with the diameter values in the windows 48.

To assist in locating the balloon adjacent the occlusion, particularly if the catheter has been withdrawn from the oesophagus and has to be re-entered into the oesophagus, graduation marks (not shown) are provided along the catheter so that a surgeon can note from the graduations the distance to which the catheter should be inserted from the mouth or nose, as the case may be, in order that the balloon is aligned with the occlusion.

Referring now to FIG. 6, there is illustrated a system according to another embodiment of the invention indicated generally by the reference numeral 50 for dilating a stricture in a lumen, for example, an occlusion in an oesophagus similar to the occlusion in the oesophagus 4 described with reference to the system of FIGS. 1 to 5. The system 50 is substantially similar to the system 1 and similar components are identified by the same reference numerals. Additionally, the system 50 comprises a device 51 also for use in dilating an occlusion in an oesophagus, which is similar to the device 5, and similar components are identified by the same reference numerals. The only difference between the system 50 and the system 1 is that in this embodiment of the invention, the voltage signals appearing on the receiving electrodes 28 are differentially applied to the analogue-to-digital converters 44 by differential op-amps 53. The inverting and non-inverting inputs of the respective op-amps 53 are coupled respectively to adjacent ones of the receiving electrodes 28, and the output of the respective op-amps 53 is applied to the analogue-to-digital converters 44. The microprocessor 35 in this embodiment of the invention is programmed to determine the transverse cross-sectional area and in turn the diameter values of the balloon 12 adjacent the corresponding receiving electrodes 28 from the voltage drop between the stimulating electrodes 25 and the adjacent receiving electrodes 28, and the differential values of the voltage signals appearing on adjacent ones of the receiving electrodes 28 from the digital values read from the analogue-to-digital converters 44.

Otherwise, the system 50 and the device 51 are similar to the system 1 and the device 5 described with reference to FIGS. 1 to 5, and the use of the system 50 and the device 51 in dilating an occlusion in an oesophagus is similar to that described with reference to the system 1 and device 5 of FIGS. 1 to 5.

Referring now to FIG. 7, there is illustrated a device indicated generally by the reference numeral 60 according to another embodiment of the invention for use with either of the systems 1 or 50 for dilating an occlusion 3 in an oesophagus 4. The device 60 is substantially similar to the device 5 and similar components are identified by the same reference numerals. The main difference between the device 60 and the device 5 is that instead of the device 60 being provided with one single balloon 12 towards its distal end 10, six balloons 61 are located in end to end abutting relationship on the catheter 8 towards the distal end 10 with the catheter 8 extending through the six balloons 61. Each balloon 61 is independently inflatable relative to the other balloons 61, and accordingly, six mutually isolated axial communicating bores 20 extend through the catheter 8 and communicate with the respective hollow interior regions 14 of the corresponding balloons 61 through corresponding radial communicating bores 21. In FIG. 7 for convenience only one of the axial communicating bores 20 is illustrated.

Additionally, within the hollow interior region 14 of each of the six balloons 61 two axially spaced apart stimulating electrodes 25 are located on the catheter 8 and one receiving electrode 28 equi-spaced axially from the stimulating electrode 25 is located between the stimulating electrodes 25. A corresponding number of mutually insulated wires 30 extend through the axial wire accommodating bore 31 in the catheter 8, and communicate with the corresponding stimulating and receiving electrodes 25 and 28 through radial wire accommodating openings 32. In this embodiment of the invention each of the six balloons 61 are identical in size and shape to each other, and each when inflated are of cylindrical configuration.

Use of the device 60 is substantially similar to use of the device 5. With the balloons 61 partially inflated, the balloons 61 are moved slowly along the oesophagus while observing the visual display screen 47 in order to identify when the central balloons 61 are axially centrally located relative to the occlusion 3. When the balloons 61 have been axially centrally located relative to the occlusion 3, the balloons 61 are further inflated with the saline solution for determining the diameter of the oesophagus 4 on the respective axially opposite ends of the occlusion 3, which is read from the visual display screen 47. The surgeon, doctor or paramedic then determines the desired diameter to which the occlusion 3 is to be dilated from the diameter of the oesophagus 4 at the axially opposite ends of the occlusion 3.

Once the desired diameter to which the occlusion 3 is to be dilated is determined, the balloons 61 which are adjacent the occlusion 3 are inflated to the diameter corresponding to the diameter to which the occlusion 3 is to be dilated, and thereafter operation of the device 60 is similar to that of the device 5.

It is envisaged, in certain cases, that it may be desirable to dilate the occlusion 3 to different diameters over its entire axial length. For example, the portions of the occlusions at the axial opposite ends thereof may be dilated to a diameter which would be greater than the diameter to which the axial central portion of the occlusion would be dilated. In which case, the balloon or balloons 61 which are adjacent the axial central portion of the occlusion 3 would be inflated to a lesser diameter than the diameter to which the balloons 61 adjacent the respective axial opposite ends of the occlusion would be inflated.

Referring now to FIG. 8, there is illustrated a device 70 according to another embodiment of the invention for use with either of the systems 1 or 50 for dilating an occlusion 3 in an oesophagus 4. The device 70 is substantially similar to the device 5, and similar components are identified by the same reference numerals. Indeed, the device 70 is substantially similar to the device 60. The device 70 comprises a plurality of independently inflatable balloons 71 which are substantially similar to the balloons 61, with the exception that in this embodiment of the invention five balloons 71 are provided, and the centre balloon 71a is of axial length greater than the axial length of the other four balloons 71b. The balloons 71b are of similar size to each other, and all five balloons 71 inflate to the same diameter. In this embodiment of the invention the centre balloon 71a is provided with a pair of stimulating electrodes 25, and four receiving electrodes 28 located between the stimulating electrodes 25. The receiving electrodes 28 are equi-spaced apart axially from each other, and the spacing between the stimulating electrodes 25 and the adjacent receiving electrodes 28 is similar to the spacing between the receiving electrodes 28.

The advantage of providing the device 70 with a centre balloon 71a which is of axial length longer than the axial length of the other balloons 71b is that it facilitates easier manoeuvring and more accurate placing of the balloons 71, and in particular, more accurate placing of the centre balloon 71a axially centrally relative to the occlusion 3. Additionally, the diameter of the centre balloon 71a can be determined at a number of axially spaced apart locations along the centre balloon corresponding to the locations of the receiving electrodes 28.

Otherwise the device 70 and its use and operation is similar to that of the devices 5 and 60.

It is envisaged that in general the systems 1 and 50 and the devices 5, 60 and 70 according to the invention will be substantially automated, and will be programmed to automatically inflate the balloon or balloons to the desired diameter to which the occlusion is to be dilated once the desired diameter has been determined and entered into the control and analysing apparatus 6.

The system 1 and the device 5 as well as the system 50 and the devices 60 and 70, and in particular, the devices 60 and 70 are also suitable for use in determining the transverse cross-sectional area and the diameter of a lumen or a cavity at a plurality of axially spaced apart locations along the lumen or cavity. To determine the diameter or cross-sectional area of a lumen or cavity using the systems 1 and 50 and the devices 5, 61 and 71, the balloon 12 or balloons 61 and 71 are located in the lumen or cavity at the locations at which the transverse cross-sectional area and/or diameter are to be determined at the axially spaced apart locations. The placing of the balloon 12 or the balloons 61 and 71 in the lumen or cavity is carried out by urging the balloon 12 or balloons 61 or 71 along a lumen or other passageway leading to the lumen or the cavity, the transverse cross-sectional area or diameter of which is to be determined, by urging the catheter therealong, as already described with reference to placing the balloon 12 in the oesophagus 4 adjacent the occlusion 3.

When in place, the balloon 12 or balloons 61 or 71 are inflated until the balloon or balloons abut an inner wall which defines the lumen or cavity. The balloon 12 or balloons 61 or 71 are inflated to a stage where the balloon or balloons tightly engage the inner wall of the lumen or cavity without dilating the lumen or cavity. As the balloon 12 or balloons 61 and 71 are being inflated, the microprocessor 35 monitors the pressure of the saline solution in the respective balloons from the pressure sensor and pressure gauge 40 or from the corresponding pressure sensors and pressure gauges 40 where more than one balloon is provided, and on a sudden increase in pressure in the saline solution, or on an increase in the rate of increase of pressure in the saline solution in the balloon 12 or the respective balloons 61 and 71, the microprocessor 35 determines that the balloon 12 or balloons 61 and 71 are in appropriate tight abutting engagement with the lumen or cavity to fill the lumen or cavity without dilating thereof.

The microprocessor 35 operates the signal generator 43 for applying the stimulating current signal to the stimulating electrodes 25 of the balloon 12 or the respective balloons 61 and 71, and the digital values of the resulting signals on the receiving electrodes 28 are read by the microprocessor 35 from the analogue-to-digital converters 44 which determines the diameter of the balloon 12 or balloons 61 and 71 adjacent the corresponding receiving electrodes 28. The diameter values are applied by the microprocessor 35 to the graphics processor 45, which in turn prepares an image representative of the inflated balloon 12 or balloons 61 and 71 on the visual display screen 47, as well as the diameters of the balloon 12 or the balloons 61 and 71 adjacent the receiving electrodes 28 along with the image representative of the inflated balloon 12 or balloons 61 or 71.

A typical image 80 displayed on the visual display screen 47 representative of the balloons 71 of the device 70 is illustrated in FIG. 9. Since the balloons 71 have been inflated to tightly abut the wall of the lumen or cavity, the image representative of the balloons 71 on the visual display screen 47 of FIG. 9 is in effect a representation of the lumen or cavity within which the balloons 71 are located, and the diameters of which are to be determined. Accordingly, the diameter values displayed on the display screen 47 in the windows 48 adjacent the image 80 are diameter values of the lumen or cavity at the locations which correspond with the locations of the receiving electrodes 28 in the balloons 71.

While the systems 1 and 50 and the devices 5, 60 and 70 have been described for use in dilating an occlusion in the oesophagus of a human subject, it will be readily apparent to those skilled in the art that the systems and the devices may be used for optionally locating and dilating an occlusion in any other lumen or hollow organ whether it be a biological organ or otherwise, and in particular, it is envisaged that the systems and the devices may be used for locating in and dilating an occlusion in the intestine or bowel of a human or animal subject, and further, may be used for locating in and dilating an occlusion in a vein or artery of a human or animal subject.

While the device 5 has been described as being provided with ten receiving electrodes, any number of receiving electrodes may be provided, and the number of receiving electrodes will largely depend on the length of the balloon, and the resolution of the image required. Additionally, while two stimulating electrodes have been provided in the balloon of the device 5, in certain cases, a single stimulating electrode may be sufficient.

It will also be appreciated that while the devices 60 and 70 have been described as comprising specific numbers of independently inflatable balloons, the devices 60 and 70 may be provided with any number of independently inflatable balloons, and it will also be appreciated that the independently inflatable balloons may be provided with more than one single receiving electrode, and the number of receiving electrodes will depend on the axial length of each independently inflatable balloon as well as the resolution required. Further, it is envisaged that in certain cases a single stimulating electrode may be sufficient in each of the independently inflatable balloons of the devices 60 and 70.

While in the embodiments of the invention the receiving electrodes have been in general described as being equi-spaced apart axially along the catheter, it is not necessary that the receiving electrodes be equi-spaced apart. The axial spacing between the receiving electrodes may vary, for example, where high resolution is required, the receiving electrodes may be located relatively closely to each other, and where lower resolution is satisfactory, the receiving electrodes may be spaced apart a greater distance from each other. Typically, it may be desirable to produce the profile of the inflated balloon adjacent the axial centre thereof with higher resolution than at the axial opposite ends, and thus, the receiving electrodes would be more closely spaced relative to each other towards the centre of the balloon, than towards the axial opposite ends, and the spacing between the receiving electrodes from the axial centre of the balloon may increase progressively towards the respective axial opposite ends of the balloon. Needless to say, if the profile of the balloon is required to be of higher resolution towards the ends than towards the centre, then the spacing between the receiving electrodes would be appropriately varied. It will also be appreciated that the spacing between the stimulating electrodes and the adjacent receiving electrodes may be different or the same as the spacing between the receiving electrodes.

While in the embodiments of the invention described the stimulating electrodes have been described as having a stimulating current signal applied thereto, it is envisaged that instead of applying a stimulating current signal to the stimulating electrodes, a stimulating voltage signal could be applied to the stimulating electrodes.

While the balloons of the devices 60 and 70 have been described as being independently inflatable, in certain cases, it is envisaged that it may not be necessary that they be independently inflatable, and in certain cases, only some of the balloons may be independently inflatable relative to others of the balloons.

While the image representative of the balloon or balloons displayed on the visual display screen has been described as being a three-dimensional image, it will readily be apparent to those skilled in the art that any suitable image representative of the balloon may be provided, for example, a longitudinal cross-sectional profile, or any other suitable image. However, it will be appreciated that the image, while it will be representative of the balloon, and may be representative of a three-dimensional image of the balloon or a longitudinal external profile of the balloon, the image may not be an exact image, since while the balloon when inflated in free air will inflate to a cylindrical configuration, nonetheless, the balloon is of a deformable material, and thus, will adopt the shape of the lumen or cavity within which it is located, which may not be entirely cylindrical, and thus, the image produced on the display screen, while it will be a reasonable representation of the balloon, will not be an exact representation of the balloon.

While the balloon and balloons have been described as being of cylindrical configuration when inflated, the balloons may be of any other shape, and may be of any other transverse cross-section besides circular. For example, the balloon or balloons when inflated may be of square, rectangular, triangular, hexagonal, polygonal or any other desired transverse cross-section, and in certain cases, it is envisaged that the transverse cross-section of the balloon or balloons may be matched to the cross-section of the lumen or cavity into which they are to be inserted. Additionally, where a plurality of adjacent balloons are provided on the catheter, the balloons may be of different transverse cross-sectional shape to each other.

While the first electrically conductive communicating means has been described as comprising a plurality of first wires coupling the stimulating and receiving electrodes to the current signal generator and the microprocessor, it is envisaged in certain cases that a single wire may be provided between the receiving electrodes and the corresponding analogue-to-digital converters, and the signals from the receiving electrodes would be multiplexed onto the single wire. The multiplexer would typically be located either in the balloon or in the catheter, and would be coupled to the receiving electrodes by individual wires. It is also envisaged that the analogue-to-digital converters may be located in the balloon or in the catheter, and a single wire would be provided from a multiplexer also located within the balloon or the catheter to the microprocessor. The multiplexer would be coupled to the analogue-to-digital converters by respective corresponding wires. Needless to say, any other suitable and convenient system for coupling the electrodes, and in particular, the receiving electrodes to the microprocessor or the analogue-to-digital converters or the op-amps may be provided.

1-147. (canceled)

148. A system for dilating a stricture in a lumen, the system comprising:
a catheter extending between a proximal end and a distal end,
an inflatable balloon defining a hollow interior region located on the catheter towards the distal end thereof with the catheter extending through the hollow interior region of the balloon, the balloon being adapted for locating in the lumen adjacent the stricture,
an inflating means for inflating the balloon with an inflating medium to dilate the stricture,
a communicating means communicating the inflating means with the hollow interior region of the balloon for accommodating the inflating medium between the inflating means and the hollow interior region of the balloon,
a pressure monitoring means for monitoring the pressure of the inflating medium in the balloon,
at least one stimulating electrode located in the hollow interior region of the balloon on one of an inner surface of the balloon and an outer surface of the catheter for receiving one of a stimulating voltage signal and a stimulating current signal,
at least one receiving electrode located in the hollow interior region of the balloon on one of the inner surface of the balloon and the outer surface of the catheter axially spaced apart from the stimulating electrode for producing a resulting signal indicative of the transverse cross-sectional area of the balloon adjacent the at least one receiving electrode in response to the one of the stimulating voltage signal and stimulating current signal when the balloon is inflated with an electrically conductive inflating medium,
a signal generator for generating the one of the stimulating voltage signal and the stimulating current signal,
a control means for operating the signal generator for producing the one of the stimulating voltage signal and the stimulating current signal, the control means being adapted for reading the resulting signal on the at least one receiving electrode in response to the one of the stimulating voltage signal and the stimulating current signal being applied to the at least one stimulating electrode, and for computing a diameter value of the balloon adjacent the at least one receiving electrode from the resulting signal read from the at least one receiving electrode,
an electrically conductive means electrically coupling the signal generator to the at least one stimulating electrode for applying the one of the stimulating voltage signal and the stimulating current signal thereto, and for electrically coupling the control means to the at least one receiving electrode for reading the resulting signal indicative of the transverse cross-sectional area of the balloon therefrom, and
a display means operable under the control of the control means for displaying the computed diameter value of the balloon adjacent the at least one receiving electrode, and
the control means being responsive to signals from the pressure monitoring means for controlling the operation of the inflating means.

149. A system as claimed in claim 148 in which a pair of the stimulating electrodes are located on the catheter axially spaced apart from each other, and a plurality of the receiving electrodes are located on the catheter between the stimulating electrodes, the receiving electrodes being axially spaced apart from each other and from the stimulating electrodes, the receiving electrodes being provided for producing resulting signals indicative of the transverse cross-sectional area of the balloon at corresponding axially spaced apart locations.

150. A system as claimed in claim 148 in which the communicating means comprises an axial communicating bore extending through the catheter from the proximal end thereof to the hollow interior region of the balloon, the inflating means being coupled to the axial communicating bore adjacent the proximal end of the catheter by a conduit.

151. A system as claimed in claim 148 in which the electrically conductive means comprises a plurality of mutually insulated electrically conductive wires extending through the catheter coupled to respective ones of the stimulating and receiving electrodes, the signal generator being coupled to the stimulating electrodes by respective corresponding ones of the wires, and the control means being coupled to the receiving electrodes by other respective corresponding ones of the wires.

152. A system as claimed in claim 151 in which the electrically conductive wires extend through an axial wire accommodating bore extending through the catheter from the proximal end thereof to the stimulating and receiving electrodes, and at least one radial wire accommodating opening extends through the catheter to the axial wire accommodating bore for accommodating the wires from the axial wire accommodating bore to the respective stimulating and receiving electrodes.

153. A system as claimed in claim 148 in which the balloon is an elongated balloon, and when inflated is of circular transverse cross-section.

154. A system as claimed in claim 148 in which the balloon defines a central balloon axis substantially coinciding with a central longitudinal axis of the catheter.

155. A system as claimed in claim 148 in which each of the stimulating and receiving electrodes comprises an electrically conductive band.

156. A system as claimed in claim 148 in which a plurality of inflatable balloons are located axially along the catheter towards the distal end thereof, at least one stimulating electrode and at least one receiving electrode axially spaced apart from the at least one stimulating electrode being located in the hollow interior region of each balloon.

157. A system as claimed in claim 148 in which the system is adapted for dilating a stricture in a hollow biological organ.

158. A system for dilating a stricture in a lumen, the system comprising:
a catheter extending between a proximal end and a distal end,
an inflatable balloon defining a hollow interior region located on the catheter towards the distal end thereof with the catheter extending through the hollow interior region of the balloon, the balloon being adapted for locating in the lumen adjacent the stricture,
an inflating means for inflating the balloon with an inflating medium to dilate the stricture,
a communicating means communicating the inflating means with the hollow interior region of the balloon for accommodating the inflating medium between the inflating means and the hollow interior region of the balloon,
a pressure monitoring means for monitoring the pressure of the inflating medium in the balloon,
at least one stimulating electrode located in the hollow interior region of the balloon on one of an inner surface of the balloon and an outer surface of the catheter for receiving one of a stimulating voltage signal and a stimulating current signal,
at least one receiving electrode located in the hollow interior region of the balloon on one of the inner surface of the balloon and the outer surface of the catheter axially spaced apart from the at least one stimulating electrode for producing a resulting signal indicative of the transverse cross-sectional area of the balloon adjacent the at least one receiving electrode in response to the one of the stimulating voltage signal and stimulating current signal when the balloon is inflated with an electrically conductive inflating medium,
a signal generator for generating the one of the stimulating voltage signal and the stimulating current signal,
a control means for operating the signal generator for producing the one of the stimulating voltage signal and the stimulating current signal, and for reading the resulting signal on the at least one receiving electrode in response to the one of the stimulating voltage signal and the stimulating current signal being applied to the at least one stimulating electrode,
an electrically conductive means electrically coupling the signal generator to the at least one stimulating electrode for applying the one of the stimulating voltage signal and the stimulating current signal thereto, and for electrically coupling the control means to the at least one receiving electrode for reading the resulting signal indicative of the transverse cross-sectional area of the balloon therefrom, and
a display means operable under the control of the control means for displaying an image representative of the balloon derived from the resulting signal read by the control means from the at least one receiving electrode, and
the control means being responsive to signals from the pressure monitoring means for controlling the operation of the inflating means.

159. A method for dilating a stricture in a lumen, the method comprising:
providing a catheter extending between a proximal end and a distal end,
providing an inflatable balloon defining a hollow interior region located on the catheter towards the distal end thereof with the catheter extending through the hollow interior region of the balloon,
providing a pressure monitoring means for monitoring the pressure of an inflating medium in the balloon,
providing at least one stimulating electrode located in the hollow interior region of the balloon on one of an inner surface of the balloon and an outer surface of the catheter,
providing at least one receiving electrode located in the hollow interior region of the balloon on one of the inner surface of the balloon and the outer surface of the catheter axially spaced apart from the at least one stimulating electrode,
providing a signal generator for generating the one of the stimulating voltage signal and the stimulating current signal,
providing a control means for operating the signal generator for producing the one of the stimulating voltage signal and the stimulating current signal and for reading the resulting signal on the at least one receiving electrode in response to the one of the stimulating voltage signal and the stimulating current signal being applied to the at least one stimulating electrode,
providing an electrically conductive means electrically coupling the signal generator to the at least one stimulating electrode and for electrically coupling the control means to the at least one receiving electrode,
providing an inflating means for inflating the balloon with an inflating medium under the control of the control means to dilate the stricture, the control means being responsive to signals from the pressure monitoring means for controlling the operation of the inflating means,
providing a communicating means communicating the inflating means with the hollow interior region of the balloon for accommodating the inflating medium between the inflating means and the hollow interior region of the balloon, the method further comprising:
entering the distal end of the catheter with the balloon thereon into the lumen until the balloon is located in the lumen with the balloon substantially centrally axially located relative to the stricture,
operating the inflating means under the control of the control means to inflate the balloon with an electrically conductive inflating medium,
applying one of a stimulating voltage signal and a stimulating current signal to the at least one stimulating electrode, and
reading a resulting signal on the at least one of the receiving electrodes adjacent the stricture for determining the transverse cross-sectional area of the balloon during inflating thereof, and
continuing inflating of the balloon by the inflating means under the control of the control means until the transverse cross-sectional area of the balloon corresponds to a desired transverse cross-sectional area to which the stricture is to be dilated.

160. A method as claimed in claim 159 in which the balloon is initially partially inflated on being inserted in the lumen, and is urged along the lumen in the partially inflated state for identifying the stricture from the resulting signal read from the at least one receiving electrode.

161. A method as claimed in claim 159 in which the balloon is urged along the lumen until the balloon is located in the general area of the stricture, and when the balloon is in the general area of the stricture, the balloon is incrementally urged along the lumen, and on each incremental movement of the balloon the balloon is inflated with the electrically conductive inflating medium and subsequently deflated, and while being inflated, the one of the stimulating voltage signal and the stimulating current signal is applied to the at least one stimulating electrode, and the resulting signal is read from the at least one receiving electrode for determining when the balloon is axially centrally located relative to the stricture.

162. A method as claimed in claim 159 in which a plurality of the balloons (12) are located on the catheter (8) towards the distal end (10) thereof with the catheter (8) extending through the balloons (12), at least one of the balloons (12) is inflated with the electrically conductive medium, and the one of the stimulating voltage signal and the stimulating current signal is applied to the at least one stimulating electrode (25) in each inflated balloon (12) and the resulting signals from the corresponding at least one receiving electrode (28) is read for determining the diameter of each inflated balloon (12) during inflating thereof, and the one or the ones of the balloons (12) adjacent the stricture (3) is inflated until the transverse cross-sectional area thereof corresponds with the desired transverse cross-sectional area to which the stricture (3) is to be dilated.

163. A device for dilating a stricture in a lumen, the device comprising:
a catheter extending between a proximal end and a distal end,
a plurality of inflatable balloons defining respective hollow interior regions located on the catheter towards the distal end thereof with the catheter extending through the hollow interior regions of the respective balloons, the balloons being adapted for locating in the lumen adjacent the stricture to dilate the stricture,
a communicating means communicating the hollow interior regions of the balloons with the exterior thereof for accommodating an inflating medium to and from the hollow interior regions of the balloons,
at least one stimulating electrode located in the hollow interior region of each balloon on one of an inner surface of the balloon and an outer surface of the catheter, and
at least one receiving electrode located in the hollow interior region of each balloon on one of the inner surface of the balloon and the outer surface of the catheter axially spaced apart from the at least one stimulating electrode of that balloon,
an electrically conductive means electrically coupled to the stimulating and receiving electrodes, and electrically accessible exteriorly of the hollow interior regions of the respective balloons, for facilitating applying one of a stimulating voltage signal and a stimulating current signal to the at least one stimulating electrode in each balloon via the electrical conducting means, and for facilitating reading of a resulting signal on the at least one receiving electrode of each balloon indicative of the transverse cross-sectional area of the balloon via the electrical conducting means, so that the transverse cross-sectional area of each inflated balloon can be monitored during inflating thereof with an electrically conductive inflating medium for determining the transverse cross-sectional area of the stricture during dilation thereof.

164. A device as claimed in claim 163 in which the respective balloons are inflatable independently of each other.

165. A device as claimed in claim 163 in which at least one of the balloons is of axial length different to the axial length of another one of the balloons.

166. A device as claimed in claim 163 in which a central one of the balloons is of axial length longer than the other balloons.

167. A device as claimed in 163 in which at least one of the balloons is of different transverse cross-sectional area when inflated to the transverse cross-sectional area of another one of the balloons when inflated.

168. A device as claimed in claim 163 in which a central one of the balloons is of transverse cross-sectional area when inflated which is greater than the transverse cross-sectional area of the other balloons when inflated.

169. A device as claimed in claim 163 in which the device is adapted for dilating a stricture in a hollow biological organ.
